SEQUENCE LISTING (5 sequences)

SEQ ID NO: 1 - DNA, 48 bp, Artificial Sequence
  primer
atatggatccggcgcgccgtcgactttttttttttttttttttttttt

SEQ ID NO: 2 - DNA, 48 bp, Artificial Sequence
  primer
atatctcgagggcgcgccggatcctttttttttttttttttttttttt

SEQ ID NO: 3 - DNA, 63 bp, Artificial Sequence
  primer
ggccagtgaattgtaatacgactcactatagggaggcggatatggatccggcgcgccgtcgac

SEQ ID NO: 4 - DNA, 24 bp, Artificial Sequence
  primer
atatctcgagggcgcgccggatcc

SEQ ID NO: 5 - DNA, 39 bp, Artificial Sequence
  primer
ggccagtgaattgtaatacgactcactatagggaggcgg

(19) United States
(12) Patent Application Publication
(10) Pub. No.: US 2008/0161197 A1
(43) Pub. Date: Jul. 3, 2008

(54) METHOD FOR AMPLIFYING MONOMORPHIC-TAILED NUCLEIC ACIDS

(76) Inventor: Kai Qin Lao, Pleasanton, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 11/955,197

(22) Filed: Dec. 12, 2007

Related U.S. Application Data
(60) Provisional application No. 60/869,669, filed on Dec. 12, 2006.

(51) Int. Cl. C40B 30/00; C12P 19/34

(52) U.S. Cl. 506/7; 435/91.2

(57) ABSTRACT

The present teachings provide methods for amplifying a plurality of target nucleic acids. In some embodiments, a first oligo-dT-universal primer comprising a 3′ oligo-dT portion and a first 5′ universal portion is used to reverse transcribe a plurality of 3′ poly-A tail-containing nucleic acids. A poly-A tail is added to the 3′ end of the first strand products to form a two-tailed reaction product. The two-tailed reaction product is amplified in a PCR, wherein the PCR comprises the first oligo-dT-universal primer, and a second oligo-dT-universal primer, wherein the second oligo-dT-universal primer comprises a 3′ oligo-dT portion and a second 5′ universal portion, wherein the second 5′ universal portion of the second oligo-dT-universal primer comprises a different sequence than the first 5′ universal portion of the first oligo-dT-universal primer. The present teachings also provide compositions and kits for amplifying target nucleic acids containing monomorphic tails.

### PRIORITY

This application claims priority under 35 U.S.C. §119(e) to U.S. Provisional Patent Application Ser. No. 60/869,669, filed Dec. 12, 2006, the entirety of which is incorporated by reference herein.

### SEQUENCE LISTING

The present application is being filed along with a Sequence Listing in electronic format. The Sequence Listing is provided as a file entitled ABIOS082A.TXT, created Dec. 12, 2007, which is 1.15 Kb in size. The information in the electronic format of the Sequence Listing is incorporated herein by reference in its entirety.

### FIELD

The present teachings are in the field of molecular and cell biology, specifically in the field of multiplexed amplifying nucleic acids that contain monomorphic tails.

### INTRODUCTION

In studies on specific tissues it is crucial to be able to directly compare both the regulator and the target being regulated. This is particularly crucial for specialized small tissue samples such as laser-dissected samples, where the specialized tissue sample represents only one or few cells. With the rapid progress in both cancer cell and stem cell research, it would be highly advantageous to be able to quantitatively profile mRNA from a single cell.

Current methods to amplify the products of gene expression involve the use of specific primer pairs for each message (see for example U.S. patent application Ser. No. 10/723,520). These gene specific pre-amplification methods require a multiplicity of specific primer pairs to cover the messages of interest. To manufacture and inventory the primer pairs for the 30,000 genes that are contained in humans, another 30,000 for mouse, etc. is expensive as well as difficult to store, retrieve, and track.

### SUMMARY

In some embodiments, the present teachings provide a method of amplifying a plurality of nucleic acids containing a monomorphic 3′ tail, comprising; hybridizing a first oligo-dX-universal primer to the monomorphic 3′ tail of the plurality of nucleic acids, wherein the first oligo-dX-universal primer comprises a 3′ oligo-dX portion and a first 5′ universal portion, and wherein the 3′ oligo-dX portion hybridizes to the monomorphic 3′ tail of the plurality of nucleic acids; extending the oligo-dX-universal primer in an extension reaction to form a plurality of first strand products comprising 3′ ends; adding a monomorphic tail to the 3′ ends of the first strand products to form a plurality of two-tailed reaction products; and, amplifying the plurality of two-tailed reaction products in a PCR, wherein the PCR comprises the first oligo-dX-universal primer, and a second oligo-dX-universal primer, wherein the second oligo-dX-universal primer comprises a 3′ oligo-dX portion and a second 5′ universal portion, wherein the second 5′ universal portion of the second oligo-dX-universal primer comprises a different sequence from the first 5′ universal portion of the first oligo-dX-universal primer, and wherein the oligo-dX portion of the second oligo-dX-universal primer comprises a nucleotide that is not complementary to the oligo-dX portion of the first oligo-dX-universal primer. Additional methods, as well as reaction compositions and kits, are also provided.

### BRIEF DESCRIPTION OF THE DRAWINGS

The skilled artisan will understand that the drawings, described below, are for illustration purposes only. The drawings are not intended to limit the scope of the present teachings in any way.

FIG. 1 depicts certain aspects of various compositions according to some embodiments of the present teachings.

FIG. 2 depicts an embodiment of a method of amplification.

### DESCRIPTION OF VARIOUS EMBODIMENTS

Aspects of the present teachings may be further understood in light of the following examples, which should not be construed as limiting the scope of the present teachings in any way. The section headings used herein are for organizational purposes only and are not to be construed as limiting the described subject matter in any way. All literature and similar materials cited in this application, including but not limited to, patents, patent applications, articles, books, treatises, and internet web pages are expressly incorporated by reference in their entirety for any purpose. When definitions of terms in incorporated references appear to differ from the definitions provided in the present teachings, the definition provided in the present teachings shall control. It will be appreciated that there is an implied “about” prior to the temperatures, concentrations, times, etc discussed in the present teachings, such that slight and insubstantial deviations are within the scope of the present teachings herein. In this application, the use of the singular includes the plural unless specifically stated otherwise. Also, the use of “comprise”, “comprises”, “comprising”, “contain”, “contains”, “containing”, “include”, “includes”, and “including” are not intended to be limiting. It is to be understood that both the foregoing general description and the following detailed description are exemplary and explanatory only and are not restrictive of the invention.

### DEFINITIONS

As used herein, the term “target nucleic acid” refers to a polynucleotide sequence that is sought to be amplified. The target nucleic can be obtained from any source, and can comprise any number of different compositional components. For example, the target nucleic acid can be DNA, RNA, transfer RNA, siRNA, and can comprise nucleic acid analogs or other nucleic acid mimics, though typically the target nucleic acids will be micro RNAs (miRNAs) and other short RNAs. The target can be methylated, non-methylated, or both. The target can be bisulfite-treated and non-methylated cytosines converted to uracil. Further, it will be appreciated that “target nucleic acid” can refer to the target nucleic acid itself, as well as surrogates thereof, for example amplification products, and native sequences. In some embodiments, the short target nucleic is a short DNA molecule derived from a degraded source, such as can be found in for example but not limited to forensics samples (see for example Butler, 2001, Forensic DNA Typing: Biology and Technology Behind STR Markers. The target nucleic acid of the present teachings can be derived from any of a number of sources, including without limitation, viruses, prokaryotes, eukaryotes, for example but not limited to plants, fungi, and animals. These sources may include, but are not limited to, whole blood, a tissue biopsy, lymph, bone marrow, amniotic fluid, hair, skin, semen, biowarfare agents, anal secretions, vaginal secretions, perspiration, saliva, buccal swabs, various environmental samples (for example, agricultural, water, and soil), research samples generally, purified samples generally, cultured cells, and lysed cells. It will be appreciated that target nucleic acids can be isolated from samples using any of a variety of procedures known in the art, for example the Applied Biosystems ABI Prism™ 6100 Nucleic Acid PrepStation, and the ABI Prism™ 6700 Automated Nucleic Acid Workstation, Boom et al., U.S. Pat. No. 5,234,809, mirVana RNA isolation kit (Ambion), etc. It will be appreciated that polynucleotides can be cut or sheared prior to analysis, including the use of such procedures as mechanical force, sonication, restriction endonuclease cleavage, or any method known in the art, to produce target nucleic acids. In general, the target nucleic acids of the present teachings will be single stranded, though in some embodiments the short target nucleic can be double stranded, and/or comprise double-stranded regions due to secondary structure, and a single strand can result from denaturation.

The primers and promoters of the present teachings can employ nucleotides as well as nucleotide analogs, including synthetic analogs having modified nucleoside base moieties, modified sugar moieties, and/or modified phosphate groups and phosphate ester moieties. Substituted ribose sugars include, but are not limited to, those riboses in which one or more of the carbon atoms, for example the 2′-carbon atom, is substituted with one or more of the same or different Cl, F, —R, —OR, —NR₂ or halogen groups, where each R is independently H, C₁-C₆ alkyl or C₅-C₁₄ aryl. Exemplary riboses include, but are not limited to, 2′-(C1-C6)alkoxyribose, 2′-(C5-C14)aryloxyribose, 2′,3′-didehydroribose, 2′-deoxy-3′-haloribose, 2′-deoxy-3′-fluororibose, 2′-deoxy-3′-chlororibose, 2′-deoxy-3′-aminoribose, 2′-deoxy-3′-(C1-C6)alkylribose, 2′-deoxy-3′-(C1-C6)alkoxyribose and 2′-deoxy-3′-(C5-C14)aryloxyribose, ribose, 2′-deoxyribose, 2′,3′-dideoxyribose, 2′-haloribose, 2′-fluororibose, 2′-chlororibose, and 2′-alkylribose, e.g., 2′-O-methyl, 4′-α-anomeric nucleotides, 1′-α-anomeric nucleotides, 2′-4′- and 3′-4′-linked and other “locked” or “LNA”, bicyclic sugar modifications (see, e.g., PCT published application nos. WO 98/22489, WO 98/39352; and WO 99/14226). Exemplary LNA sugar analogs within a polynucleotide include, but are not limited to, the structures:

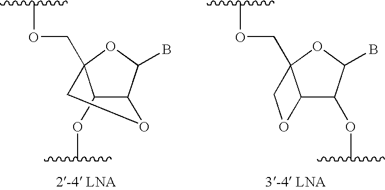

where B is any nucleotide base.

Modifications at the 2′- or 3′-position of ribose include, but are not limited to, hydrogen, hydroxy, methoxy, ethoxy, allyloxy, isopropoxy, butoxy, isobutoxy, methoxyethyl, alkoxy, phenoxy, azido, amino, alkylamino, fluoro, chloro and bromo. Nucleotides include, but are not limited to, the natural D optical isomer, as well as the L optical isomer forms (see, e.g., Garbesi (1993) Nucl. Acids Res. 21:4159-65; Fujimori (1990) J. Amer. Chem. Soc. 112:7435; Urata, (1993) Nucleic Acids Symposium Ser. No. 29:69-70). When the nucleotide base is purine, e.g. A or G, the ribose sugar is attached to the N⁹-position of the nucleotide base. When the nucleotide base is pyrimidine, e.g. C, T or U, the pentose sugar is attached to the N¹-position of the nucleotide base, except for pseudouridines, in which the pentose sugar is attached to the C5 position of the uracil nucleotide base (see, e.g., Kornberg and Baker, (1992) DNA Replication, 2^{nd} Ed., Freeman, San Francisco, Calif.).

One or more of the pentose carbons of a nucleotide may be substituted with a phosphate ester having the formula:

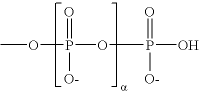

where α is an integer from 0 to 4. In certain embodiments, α is 2 and the phosphate ester is attached to the 3′- or 5′-carbon of the pentose. In certain embodiments, the nucleotides are those in which the nucleotide base is a purine, a 7-deazapurine, a pyrimidine, or an analog thereof. “Nucleotide 5′-triphosphate” refers to a nucleotide with a triphosphate ester group at the 5′ position, and are sometimes denoted as “NTP”, or “dNTP” and “ddNTP” to particularly point out the structural features of the ribose sugar. The triphosphate ester group may include sulfur substitutions for the various oxygens, e.g. α-thio-nucleotide 5′-triphosphates. For a review of nucleotide chemistry, see: Shabarova, Z. and Bogdanov, A. Advanced Organic Chemistry of Nucleic Acids, VCH, New York, 1994.

As used herein, the term “hybridization” refers to the complementary base-pairing interaction of one nucleic acid with another nucleic acid that results in the formation of a duplex, triplex, or other higher-ordered structure, and is used herein interchangeably with “annealing.” Typically, the primary interaction is base specific, e.g., A/T and G/C, by Watson/Crick and Hoogsteen-type hydrogen bonding. Base-stacking and hydrophobic interactions can also contribute to duplex stability. Conditions for hybridizing primers to complementary and substantially complementary target sequences are well known, e.g., as described in Nucleic Acid Hybridization, A Practical Approach, B. Hames and S. Higgins, eds., IRL Press, Washington, D.C. (1985) and J. Wetmur and N. Davidson, Mol. Biol. 31:349 et seq. (1968). In general, whether such annealing takes place is influenced by, among other things, the length of the polynucleotides and the complementary, the pH, the temperature, the presence of mono- and divalent cations, the proportion of G and C nucleotides in the hybridizing region, the viscosity of the medium, and the presence of denaturants. Such variables influence the time required for hybridization. Thus, the preferred annealing conditions will depend upon the particular application. Such conditions, however, can be routinely determined by the person of ordinary skill in the art without undue experimentation. It will be appreciated that complementarity need not be perfect; there can be a small number of base pair mismatches that will minimally interfere with hybridization between the target sequence and the primers of the present teachings. However, if the number of base pair mismatches is so great that no hybridization can occur under minimally stringent conditions then the sequence is generally not a complementary target sequence. Thus, complementarity herein is meant that primers are sufficiently complementary to the target sequence to hybridize under the selected reaction conditions to achieve the ends of the present teachings. Likewise, the immobilized probes on the solid support are sufficiently complementary to the in vitro transcription products to hybridize under the selected reaction conditions to achieve the ends of the present teachings.

The term “corresponding” as used herein refers to a specific relationship between the elements to which the term refers. Some non-limiting examples of corresponding include a first oligo-dT-universal primer corresponds with a collection of poly-A containing messenger RNAs, etc.

As used herein, the term “first oligo-dT-universal primer” refers to a nucleic acid molecule that contains an extendable 3′OH group, and which contains a 3′ oligo-dT portion and a first 5′ universal portion. The 3′ oligo-dT portion can hybridize to the 3′ poly-A tail of a 3′ poly-A tail-containing nucleic acid, such as a messenger RNA.

As used herein, the term “second oligo-dT-universal primer” refers to a nucleic acid molecule that contains an extendable 3′OH group, and which contains a 3′ oligo-dT portion and a second 5′ universal portion. The 3′ oligo-dT portion can hybridize to the tailed portion of the first strand product. For example, when a poly-A polymerase is used to add a poly-A to a first strand product to form a two-tailed reaction product, the 3′ oligo-dT portion of the second oligo-dT-universal primer can hybridize to this added poly-A tail. The second 5′ universal portion of the second oligo-dT-universal primer comprises a different sequence than the first 5′ universal portion of the first oligo-dT-universal primer.

As used herein, the term “array” refers to any of a variety of solid-support based apparati for detecting target polynucleotides. In some embodiments, array can be a ‘microarray,’ and can contain a plurality of elements, each of which contains a particular probe nucleic acid that can hybridized to a corresponding target nucleic acid. Illustrative microarrays are the Applied Biosystems 1700 Chemiluminescent Microarray Analyzer and other commercially available array systems available from Affymetrix, Agilent, Illumina, and Amersham Biosciences, among others (see also Gerry et al., J. Mol. Biol. 292:251-62, 1999; De Bellis et al., Minerva Biotec 14:247-52, 2002; and Stears et al., Nat. Med. 9:140-45, including supplements, 2003). It will also be appreciated that detection can comprise reporter groups that are incorporated into the reaction products, for example due to the incorporation of labeled dNTPs during an in vitro amplification, or attached to reaction products, for example but not limited to the inclusion DIG-labeled dUTP (Digoxigenin-labeled dUTP) in the reaction, with subsequent labeling with alkaline-phosphatase-based chemiluminescence. Some illustrative detection methods are further described in U.S. Pat. No. 6,905,826.

As used herein, the term “first oligo-dX-universal primer” refers to a nucleic acid molecule that contains an extendable 3′ OH group, and which comprises a 3′ oligo-dX portion and a first 5′ universal portion. The 3′ oligo-dX portion hybridizes to the monomorphic 3′ tail of a plurality of target nucleic acids, such as the poly-A tail of messenger RNAs. The X refers to the notion that the nucleotide can be a monomorphic run of any of A, T, G, C, or an appropriate analog. Thus, when the oligo-dX portion hybridizes to the poly-A tail of messenger RNA, the X can be a T, and there is thus a monomorphic run of T residues (e.g. 20 consecutive T residues).

As used herein, the term “second oligo-dX-universal primer” refers to nucleic acid molecule that contains an extendable 3′OH group, and which comprises a 3′ oligo-dX portion and a second 5′ universal portion. The second 5′ universal portion of the second oligo-dX-universal primer comprises a different sequence from the first 5′ universal portion of the first oligo-dX-universal primer. The X refers to the notion that the nucleotide can be any of A, T, G, C, or an appropriate analog. Thus, when the oligo-dX portion hybridizes to the poly-A tail added by a poly-A polymerase to a first strand product, the X can be a T, and there is thus a monomorphic run of T residues (e.g. 20 consecutive T residues). The oligo-dX portion of the second oligo-dX-universal primer comprises a nucleotide that is not complementary to the oligo-dX portion of the first oligo-dX-universal primer.

In saying that the second 5′ universal portion of the second oligo-dX-universal primer comprises a different sequence from the first 5′ universal portion of the first oligo-dX-universal primer, it will be appreciated that the sequence can vary by at least 6 nucleotides, and in some embodiments can vary by at least 10 nucleotides, can vary by at least 15 nucleotides, and/or can vary by at least 20 nucleotides.

As used herein, the term “two-tailed reaction product” refers to a nucleic molecule that contains a monomorphic nucleotide sequence on both of its ends. For example, a first strand synthesis product can contain a collection of T's at it's 5′ end (resulting from the incorporation of the dT-containing reverse primer that hybridized to the poly-A tail of the original messenger RNA), and can also contain a collection of A's at it's 3′ end (resulting from the incorporation of the A's by a poly-A polymerase).

### Exemplary Embodiments

In some embodiments, the present teachings provide a method of using anchored universal primers to amplify the entire population of mRNAs from a single small sample on the order of one or a few cells from any eukaryote. The amplified mRNA products produced by this strategy can be readily assayed by micro-array platforms, such as AB1700, and/or real time PCR platforms commercially available from Applied Biosystems.

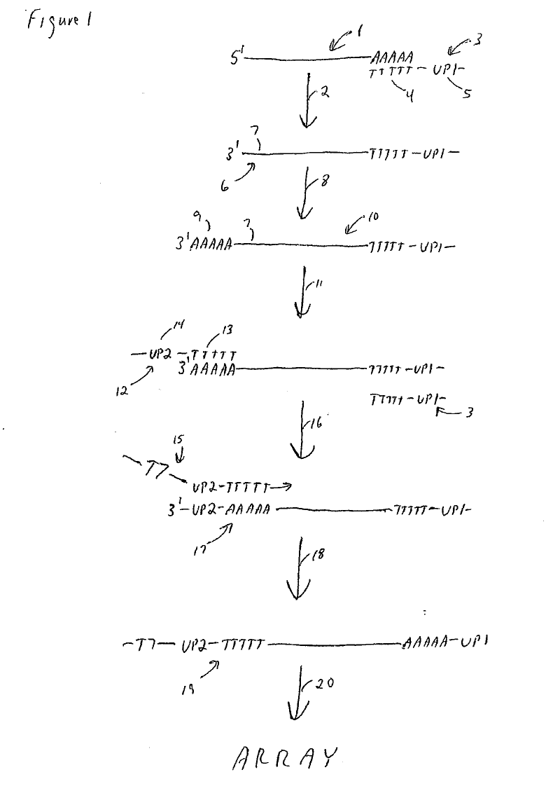

For example, as depicted in FIG. 1, a poly-A tail-containing nucleic acid (1) is reverse transcribed (2) with a first oligo-dT-universal primer (3). The first oligo-dT-universal primer (3) contains a 3′ oligo-dT portion (4) and a first 5′ universal portion (UP1, 5). The reverse transcription (2) results in a first strand product (6) comprising a 3′ end (7). An addition reaction can be performed (8) in which a poly-A tail (9) is added to the 3′ end of the first strand product (7), to form a two-tailed reaction product (10). The two-tailed reverse transcription reaction product can be amplified in a PCR (11). The PCR comprises the first oligo-dT universal primer (3) and a second oligo-dT universal primer (12). The second oligo-dT-universal primer (12) comprises a 3′ oligo-dT portion (13) and a second 5′ universal portion (UP2, 14). Of note, the second 5′ universal portion of the second oligo-dT-universal primer (14) comprises a different sequence than the first 5′ universal portion of the first oligo-dT-universal primer (5).

In some embodiments, thereafter, a promoter-linked primer (15) can be hybridized (16) to one of the strands of the PCR product (17). An extension reaction (18) results in a promoter-containing product (19), which can be amplified in an in vitro transcription transcription reaction (20), with subsequent array analysis.

Thus, in some embodiments, the present teachings provide a method of amplifying a 3′ poly-A tail-containing nucleic acid in a sample comprising; a) hybridizing a first oligo-dT-universal primer to the poly-A tail of the 3′ poly-A tail-containing nucleic acid, wherein the first oligo-dT-universal primer comprises a 3′ oligo-dT portion and a first 5′ universal portion, and wherein the 3′ oligo-dT portion hybridizes to the 3′ poly-A tail of the 3′ poly-A tail-containing nucleic acid; b) extending the oligo-dT-universal primer in an extension reaction to form a first strand product comprising a 3′ end; c) adding a poly-A tail to the 3′ end of the first strand product to form a two-tailed reaction product; and, d) amplifying the two-tailed reaction product in a PCR to form an amplified 3′ poly-A tail-containing nucleic acid, wherein the PCR comprises the first oligo-dT-universal primer, and a second oligo-dT-universal primer, wherein the second oligo-dT-universal primer comprises a 3′ oligo-dT portion and a second 5′ universal portion, wherein the second 5′ universal portion of the second oligo-dT-universal primer comprises a different sequence than the first 5′ universal portion of the first oligo-dT-universal primer. In some embodiments, the method further comprises truncating the 3′ end of the first strand product with an exonuclease between b) and c). In some embodiments, the truncating comprises removing at least 100 nucleotides. In some embodiments, the 3′ oligo-dT portion of the first oligo-dT-universal primer comprises at least 20 dT's, and wherein the oligo-dT portion of the second oligo-dT-universal primer comprises at least 20 dT's. In some embodiments, the first 5′ universal portion of the first oligo-dT-universal primer comprises at least 10 nucleotides, and wherein the second 5′ universal portion of the second oligo-dT-universal primer comprises at least 10 nucleotides. In some embodiments, the sample comprises the entire transcriptome of 3′ poly-A tail-containing nucleic acids, the method further resulting in a plurality of amplified 3′ poly-A tail-containing nucleic acids.

In some embodiments, the amplified 3′ poly-A tail-containing nucleic acids are further amplified in a primer-extension reaction comprising a promoter-linked primer to form a plurality of promoter-containing products, the method further comprising in vitro transcription of the promoter-containing products to form a plurality of in vitro transcription products, wherein the in vitro transcription comprises at least one labeled nucleotide. Methods employing promoter sequences to effectuate in vitro transcription are known, and can be found for example in U.S. Pat. No. 5,514,545, U.S. Pat. No. 5,545,522, U.S. Pat. No. 5,554,552, U.S. Pat. No. 5,716,785, U.S. Pat. No. 5,891,636, and U.S. Pat. No. 6,114,152. In some embodiments, the plurality of in vitro transcription products are analyzed on an array. In some embodiments, the first oligo-dT-universal primer or the second oligo-dT-universal primer further contains a promoter, the method further comprising in vitro transcription of the amplified 3′ poly-A tail-containing nucleic acid by a promoter-recognizing enzyme to form a plurality of in vitro transcription products, wherein the in vitro transcription comprises at least one labeled nucleotide. In some embodiments, the plurality of in vitro transcription products are analyzed on an array.

More generally, the present teachings provide a method of amplifying a plurality of nucleic acids containing a monomorphic 3′ tail, comprising; hybridizing a first oligo-dX-universal primer to the monomorphic 3′ tail of the plurality of nucleic acids, wherein the first oligo-dX-universal primer comprises a 3′ oligo-dX portion and a first 5′ universal portion, and wherein the 3′ oligo-dX portion hybridizes to the monomorphic 3′ tail of the plurality of nucleic acids; extending the oligo-dX-universal primer in an extension reaction to form a plurality of first strand products comprising 3′ ends; adding a monomorphic tail to the 3′ ends of the first strand products to form a plurality of two-tailed reaction products; and, amplifying the plurality of two-tailed reaction products in a PCR, wherein the PCR comprises the first oligo-dX-universal primer, and a second oligo-dX-universal primer, wherein the second oligo-dX-universal primer comprises a s3′ oligo-dX portion and a second 5′ universal portion, wherein the second 5′ universal portion of the second oligo-dX-universal primer comprises a different sequence from the first 5′ universal portion of the first oligo-dX-universal primer, and wherein the oligo-dX portion of the second oligo-dX-universal primer comprises a nucleotide that is not complementary to the oligo-dX portion of the first oligo-dX-universal primer. In some embodiments, the monomorphic tail of the 3′ ends of the first strand products comprise the same nucleotide as the monomorphic 3′ tail of the plurality of nucleic acids. In some embodiments, the monomorphic tail of the 3′ ends of the first strand products comprise adenine, and the monomorphic 3′ tail of the plurality of nucleic acids comprise adenine.

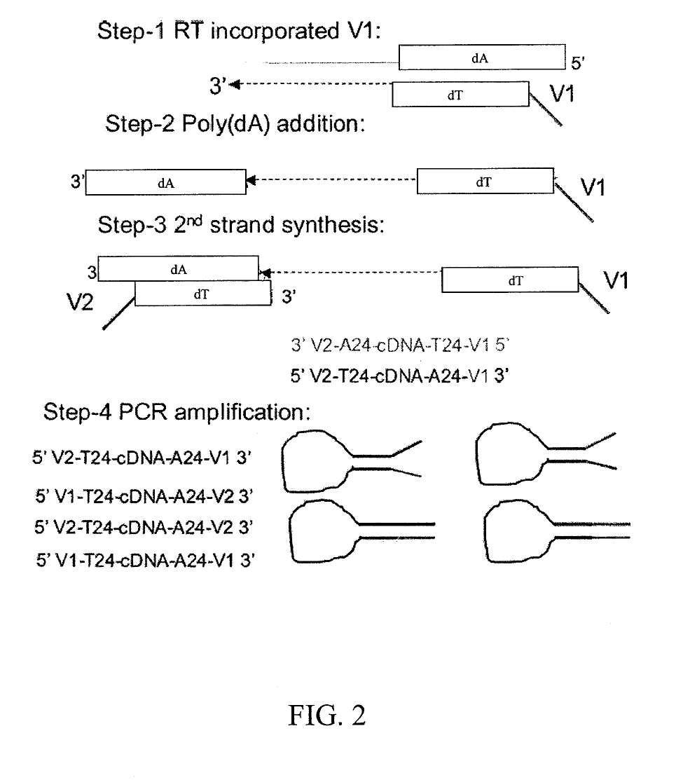

In some embodiments, the monomorphic tail on each end of the two-tailed reaction product allows for the product to self-hybridize, thereby forming a looped structure, which can be useful in eliminating primer dimer amplification in later steps. An example of such an embodiment is depicted in FIG. 2. In the method depicted in FIG. 2, one first incorporates a first primer that has a dT portion and a first universal portion (V1). This first primer can be added in a reverse transcription reaction (e.g., RT incorporated V1). Following this, the product of the above step can have a dA portion added (step 2). In step 3, second strand synthesis can occur using a second primer that has a dT section to bind to the dA portion added in step 2. Following this, a PCR amplification can be performed on the product from step 3. As noted in FIG. 2, the products from step 3, because of the dX sections, will be able to form looped structures (a variety of possibilities are depicted in FIG. 2). Those loops that are large enough to allow efficient internal PCR amplification, will allow the target DNA within the loop to be amplified. The smaller looped structures (such as those produced by primer dimers or those that resulted from internal spurious priming) will be amplified relatively less or not at all.

An exemplary protocol for performing one embodiment of the present teachings was performed as follows. Aspects of the present teachings may be further understood in light of the following example, which should not be construed as limiting the scope of the teachings in any way.

Single Cell cDNA Protocol for Microarray Quality

10 mature oocytes (VAS male×F1 female. Isolate mature oocytes from the oviduct. Sample 11 is picking buffer only control)

| | | | Volume/ | Final | | | | | |
|---|---|---|---|---|---|---|---|---|---|
| (1) Cell lysis buffer | Stock | | Total RT | Concentration | | 1X | | 12X | |
| (4.05 ul/tube) | Concentration | | Volume (5 ul) | in RT (5 ul) | | Volume | | Volume | |
| 10xPCR buffer II | 10 X | | 0.09 | 0.9X | | 0.45 | ul | 5.4 | ul |
| 25 mM MgCl2 | 25 | mM | 0.054 | 1.35 | mM | 0.27 | ul | 3.24 | ul |
| 10% NP40 | 10% | | 0.045 | 0.45% | | 0.225 | ul | 2.7 | ul |
| 0.1M DTT | 100 | mM | 0.045 | 4.5 | mM | 0.225 | ul | 2.7 | ul |
| Prime RNase Inhibitor | 30 | U/ul | 0.009 | 0.27 | U/ul | 0.045 | ul | 0.54 | ul |
| RNAguard RNase | 20-40 | U/ul | 0.009 | 0.18-0.36 | U/ul | 0.045 | ul | 0.54 | ul |
| Inhibitor (HP) | | | | 12.5 | nM | | | | |
| 0.5 uM V1-T24 primer | 500 | nmol/L | 0.025 | (0.18 ng/ul) | | 0.125 | ul | 1.5 | ul |
| (7.35 ng/ul) | | | | | | | | | |
| 2.5 mM dNTP | 2.5 | mM | 0.018 | 0.045 | mM | 0.09 | ul | 1.08 | ul |
| H₂O | — | | 0.522 | — | | 2.575 | ul | 30.9 | ul |
| Total Volume | | | 0.81 | | | 4.05 | ul | 48.6 | ul |

### Step 1: Cell Lysis

Dilute V1-T24 Primer: 1 ul 100 uM V1-T24 Prime+199 ul H₂O to get 0.5 uM V1-T24 Primer.
Seed single cell (With 0.5 μl PBS) in to each 0.5-ml thin-wall PCR tube containing Cell Lysis Buffer (1) 4.05 μl.
Centrifuge for 15 sec at 10,000 rpm. And put on ice immediately.
Incubate at 70° C. for 90 sec. and put on ice immediately.
Centrifuge tubes briefly and put them on ice immediately for 1 min.

After this step, all mRNA are released.

| | | | Volume/ | Final | | | | |
|---|---|---|---|---|---|---|---|---|
| (2) RT mix | Stock | | Total RT | Concentration | | 1X | 20X | |
| (0.45 ul/tube) | Concentration | | Volume (5 ul) | in RT (5 ul) | | Volume | Volume | |
| SuperScript III Reverse | 200 | U/ul | 0.066 | 13.2 | U/ul | 0.33 ul | 6.6 | ul |
| Transcriptase | | | | | | | | |
| RNAguard RNase | 20-40 | U/ul | 0.01 | 0.2-0.4 | U/ul | 0.05 ul | 1 | ul |
| Inhibitor (HP) | 1-10 | ug/ul | | | | | | |
| T4 gene 32 protein | (5 ug/ul in average) | | 0.014 | 0.07 | U/ul | 0.07 ul | 1.4 | ul |
| Total Volume | | | 0.09 | | | 0.45 ul | 9 | ul |

T4 Gene 32 Protein is a single-strand specific DNA binding protein. It is reported to improve the yield of long PCR products when used at a concentration of 0.5 to 1 nmol during PCR. T4 Gene 32 Protein has been used to stimulate in vitro DNA synthesis and to stabilize single-stranded regions of DNA for site specific mutagenesis.

### Step 2: Reverse Transcription

Add 0.45 μl RT mix (2) to each tube.
Total Volume: 5 ul
Incubate at 50° C. for 10 min.
Inactivate the enzyme at 70° C. for 10 min.
Centrifuge tubes briefly and put them on ice immediately for 1 min.

After this step, get first strand cDNA for all mRNA.

| | | Volume/Total | Final | | | |
|---|---|---|---|---|---|---|
| (3) Exonuclease I mix | Stock | Cut Volume | Concentration | 1X | 15X | |
| (1.0 ul/tube) | Concentration | (1 ul) | in Cut (1 ul) | Volume | Volume | |
| 10x Exonuclease I buffer | 10 X | 0.1 | 1 X | 0.1 ul | 1.5 | ul |
| H₂O | — | 0.8 | — | 0.8 ul | 12 | ul |
| Exonuclease I (Takara) | 5 U/ul | 0.1 | 0.5 U/ul | 0.1 ul | 1.5 | ul |
| Total Volume | | 1 | | 1 ul | 15 | ul |

### Step 3: Unreacted-Primer Digestion

Add 1.0 μl Exonuclease I (3) to each tube.
Total Volume: 6 ul
Incubate at 37° C. for 30 min (using PCR machine).
Inactivate the enzyme at 80° C. for 25 min (using PCR machine).
Centrifuge tubes briefly and put them on ice immediately for 1 min.

After this step, all free V1-T24 primers were destroyed and the 3′-end of cDNAs were shortened about 100 bp. But the 5′-end of cDNAs (V1-T24 sequence) are intact.

| | | | Volume/Total | Final | | | | | |
|---|---|---|---|---|---|---|---|---|---|
| (4) TdT mix | Stock | | Tailing Volume | Concentration | | 1X | | 12X | |
| (6.0 ul/tube) | Concentration | | (6 ul) | in Tailing (6 ul) | | Volume | | Volume | |
| 10 x PCR buffer II | 10 X | | 0.1 | 1 X | | 0.6 | ul | 7.2 | ul |
| 25 mM MgCl2 | 25 | mM | 0.06 | 1.5 | mM | 0.36 | ul | 4.32 | ul |
| 100 mM dATP (Promega) | 100 | mM | 0.03 | 3 | mM | 0.18 | ul | 2.16 | ul |
| H₂O | — | | 0.71 | — | | 4.26 | ul | 51.12 | ul |
| TdT | 15 | U/ul | 0.05 | 0.75 | U/ul | 0.3 | ul | 3.6 | ul |
| RNase H (Roche) | 5 | U/ul | 0.05 | 0.1 | U/ul | 0.3 | ul | 3.6 | ul |
| Total Volume | | | 1 | | | 6 | ul | 72 | ul |

### Step 4: Poly(dA) Addition

Add 6-μl TdT mixture (4) to each tube.
Total Volume: 12 ul
Incubate at 37° C. for 15 min.
Inactivate the enzyme at 70° C. for 10 min.
Centrifuge tubes briefly and put them on ice immediately for 1 min.

After this step, 3′-end of the first-stranded cDNAs has a poly(A) tail now.

| | | | Volume/Total | Final | | | | | |
|---|---|---|---|---|---|---|---|---|---|
| (5) PCR mixture 1 | Stock | | PCR Volume | Concentration | | 1X | | 45X | |
| (19 ul x 4/RT product) | Concentration | | (19 ul) | in PCR (19 ul) | | Volume | | Volume | |
| 10xExTaq buffer | 10 X | | 0.1 | 1 X | | 1.9 | ul | 85.5 | ul |
| 2.5 mM dNTP | 2.5 | mM | 0.1 | 0.25 | mM | 1.9 | ul | 85.5 | ul |
| | | | | 0.004 | ug/ul | | | | |
| 1 ug/ul V3-T24 primer | 100 | umol/L | 0.003 | (0.3 uM) | | 0.057 | ul | 2.565 | ul |
| (100 uM) | | | | | | | | | |
| H₂O | — | | 0.786 | — | | 14.953 | ul | 672.885 | ul |
| ExTaq Hot Start Version | 5 | U/ul | 0.01 | 0.05 | U/ul | 0.19 | ul | 8.55 | ul |
| Total Volume | | | 1 | | | 19 | ul | 855 | ul |

Step 5: 2nd-Strand Synthesis
Divide the poly-dA tailed RT product (12 μl) into four empty thin-wall-200-μl-PCR tubes (3 μl/tube).
Add 19-μl PCR mix 1 (primer V3-T24) (5) in each tube. (Final Concentration of V3-T24 Primer: 0.26 uM)
Total Volume: 22 ul
Do 1-cycle PCR:
95° C. for 3 min, 50° C. for 2 min, and 72° C. for 6 min (for 1 cycle).
Put tubes on ice for 1 min.
Centrifuge tubes briefly and put them on ice immediately.

After this step, the second-strand cDNAs are 5′-V3-T24-cDNA-A24-V1-3′

| | | | Volume/Total | Final | | | | | |
|---|---|---|---|---|---|---|---|---|---|
| (6) PCR mixture 2 | Stock | | PCR Volume | Concentration | | 1X | | 45X | |
| (19 ul x 4/RT product) | Concentration | | (19 ul) | in PCR (19 ul) | | Volume | | Volume | |
| 10xExTaq buffer | 10 X | | 0.1 | 1 X | | 1.9 | ul | 85.5 | ul |
| 2.5 mM dNTP | 2.5 | mM | 0.1 | 0.25 | mM | 1.9 | ul | 85.5 | ul |
| 100 uM V1-T24 primer | | | | 2.2 | uM | | | | |
| (0.74 ug/ul) | 100 | umol/L | 0.022 | (0.016 ug/ul) | | 0.418 | ul | 18.81 | ul |
| 100 uM V3-T24 primer | | | | 2.2 | uM | | | | |
| (0.74 ug/ul) | 100 | umol/L | 0.022 | (0.016 ug/ul) | | 0.418 | ul | 18.81 | ul |
| H₂O | — | | 0.754 | — | | 14.174 | ul | 637.83 | ul |
| ExTaq Hot Start Version | 5 | U/ul | 0.01 | 0.05 | U/ul | 0.19 | ul | 8.55 | ul |
| Total Volume | | | 1 | | | 19 | ul | 855 | ul |

### Step 6: 18 Cycle PCR

Add 19 μl PCR mix 2 (1 ug/ul of primer V1-T24 & Primer V3-T24) (6) in each tube.
Total Volume: 41 ul. (Final Concentration of V1-T24 and V3-T24 Primer: 1 uM)
Do 18-cycle PCR:
95° C. for 30 sec, 67° C. for 1 min, and 72° C. for 6 min (+6 sec for each cycle) (for 18 cycles).

After this step, all cDNAs were amplified.

### Step 7: DNA Purification

Mix the divided PCR product together (164 ul for each sample). Aliquot 20 ul for PCR check quality. The left 140 ul is for purification.
Purify DNA with QIAquick PCR Purification Kit, and elute with 50 μl EB buffer.
Store at −80° C.

| | | | Volume/Total | Final | | | | | |
|---|---|---|---|---|---|---|---|---|---|
| (7) PCR mix for T7 | Stock | | PCR Volume | Concentration | | 1X | | 45X | |
| promoter addition | Concentration | | (42 ul) | in PCR (42 ul) | | Volume | | Volume | |
| 10 x ExTaq Buffer | 10 X | | 0.1 | 1 X | | 4.2 | ul | 189 | ul μl |
| 2.5 mM dNTP | 2.5 | mM | 0.1 | 0.25 | mM | 4.2 | ul | 189 | ul μl |
| | | | | 0.3 | uM | | | | |
| 100 uM T7-V1 Primer | 100 | umol/L | 0.003 | (0.0195 ug/ul) | | 0.126 | ul | 5.67 | ul μl |
| (1.95 ug/ul) | | | | | | | | | |
| H₂O | — | | 0.787 | — | | 33.054 | ul | 1487.43 | μl |
| ExTaq Hot start | 5 | U/ul | 0.01 | 0.05 | U/ul | 0.42 | ul | 18.9 | μl |
| Total Volume | | | (42 μl x 4) | | | 42 | ul | 1890 | μl |

### Step 8: 9 Cycle PCR for T7 Promoter Addition

Add 1.2 μl of the 18-cycle PCR product (step 7 product) to each of four empty thin-wall-200-μl PCR tubes (4×1.2 μl=4.8 μl in total).
Add 42 μl PCR mixture (7) to each tube. (Final Concentration of T7-V1: 0.29 uM)
Total Volume: 43.2 ul
Do 1 cycle of PCR;
95° C. for 3 min, 64° C. for 1 min, 72° C. for 6 min (for 1 cycle),

After this step, The cDNAs are 5′-V3-T24-cDNA-A24-V1-T7-340

Or: 5′-T7-V1-T24-cDNA-A24-V3-3′

| | | | Volume/Total | Final | | | | | |
|---|---|---|---|---|---|---|---|---|---|
| (8) PCR mix for T7 | Stock | | PCR Volume | Concentration | | 1X | | 45X | |
| promoter addition | Concentration | | (44 ul) | in PCR (44 ul) | | Volume | | Volume | |
| 10 x ExTaq Buffer | 10 X | | 0.1 | 1 X | | 4.4 | ul | 198 | ul μl |
| 2.5 mM dNTP | 2.5 | mM | 0.1 | 0.25 | mM | 4.4 | ul | 198 | ul μl |
| | | | | 2 | uM | | | | |
| 100 uM T7 Primer | 100 | umol/L | 0.02 | (0.024 ug/ul) | | 0.88 | ul | 39.6 | ul μl |
| (1.21 ug/ul) | | | | 2 | uM | | | | |
| 100 uM V3 Primer | 100 | umol/L | 0.02 | (0.0148 ug/ul) | | 0.88 | ul | 39.6 | μl |
| (0.74 ug/ul) | | | | | | | | | |
| H₂O | — | | 0.75 | — | | 33 | ul | 1485 | μl |
| ExTaq Hot start | 5 | U/ul | 0.01 | 0.05 | U/ul | 0.44 | ul | 19.8 | μl |
| Total Volume | | | (44 μl x 4) | | | 44 | ul | 1980 | μl |

### Step 9: 9 Cycle PCR for T7 Promoter Addition

Add 44 μl PCR mixture (8) to each tube. (Final Concentration of T7 and V3 Primer: 1 uM; Final Concentration of T7-V1 Primer: 0.15 uM)
Total Volume: 87.2 ul
Do 9 cycles of PCR;
95° C. for 3 min (for 1 cycle),
95° C. for 30 sec, 67° C. for 1 min, and 72° C. for 6 min (+6 sec for each cycle) (for 9 cycles).

After this step, The cDNAs are 5′-V3-T24-cDNA-A24-V1-T7-3′

Or: 5′-T7-V1-T24-cDNA-A24-V3-3′

### Step 9: DNA Purification-2

Mix the divided PCR product together (348.8 ul for each sample).
Purify using QIAquick PCR purification Kit, and elute with 30 μl EB buffer.
Store at −80° C.

### Step 10: Gel Purification

Add 6 μl 6×DNA loading Buffer to 30 μl-Step 9 product.
Electrophoresis the samples with 2% agarose gel until the BPB dye moves for 2 cm (for 10 min at the constant voltage of 100 v) (The agarose gel should be made thin to increase recovery from the gel.).
Recover the product cDNA smear larger than 300 bps by razors (0.2-0.4 g).
Purify the cDNA with QIAquick Gel Extraction Kit, and elute with 35 μl EB buffer.
Store at −80° C.

It will be appreciated that step 8 will incorporate T7 sequence into the template for the IVT step that a microarray system, such as the AB1700, can use to incorporate Dig-dUTP for subsequent chemiluminescent detection. This step will not amplify the cDNA templates.

The following PCR step (step 9) is optional, and is not necessary. It's inclusion will depend, for example, on how many fold amplification is needed for a particular application.

Sequences employed in this example were as follows:

SEQ ID NO: 1 V1(dT)24
ATATGGATCCGGCGCGCCGTCGACTTTTTTTTTTTTTTTTTTTTTTTT
SEQ ID NO: 2 V3(dT)24
ATATCTCGAGGGCGCGCCGGATCCTTTTTTTTTTTTTTTTTTTTTTTT
SEQ ID NO: 3 T7V1
GGCCAGTGAATTGTAATACGACTCACTATAGGGAGGCGGATATGGATCCG
GCGCGCCGTCGAC
SEQ ID NO: 4 V3-primer
ATATCTCGAGGGCGCGCCGGATCC
SEQ ID NO: 5 T7-primer
GGCCAGTGAATTGTAATACGACTCACTATAGGGAGGCGG

### Kits

In certain embodiments, the present teachings also provide kits designed to expedite performing certain methods. In some embodiments, kits serve to expedite the performance of the methods of interest by assembling two or more components used in carrying out the methods. In some embodiments, kits may contain components in pre-measured unit amounts to minimize the need for measurements by end-users. In some embodiments, kits may include instructions for performing one or more methods of the present teachings. In certain embodiments, the kit components are optimized to operate in conjunction with one another.

Thus, in some embodiments the present teachings provide a kit for amplifying a 3′ poly-A tail-containing nucleic acid in a sample comprising; a) a first oligo-dT-universal primer, wherein the first oligo-dT-universal primer comprises a 3′ oligo-dT portion and a first 5′ universal portion, and wherein the 3′ oligo-dT portion hybridizes to the 3′ poly-A tail of a 3′ poly-A tail-containing nucleic acid; b) a means for adding a poly-A tail to the 3′ end of a first strand product; and, c) a second oligo-dT-universal primer, wherein the second oligo-dT-universal primer comprises a 3′ oligo-dT portion and a second 5′ universal portion, wherein the second 5′ universal portion of the second oligo-dT-universal primer comprises a different sequence than the first 5′ universal portion of the first oligo-dT-universal primer. In some embodiments, the means for adding a poly-A tail to the 3′ end of the first strand product comprises a poly-A polymerase.

More generally, the present teachings provide a kit for amplifying a plurality of nucleic acids containing a first monomorphic 3′ tail, comprising; (a) a first oligo-dX-universal primer complementary to the monomorphic 3′ tail of the plurality of nucleic acids, wherein the first oligo-dX-universal primer comprises a 3′ oligo-dX portion and a first 5′ universal portion, and wherein the 3′ oligo-dX portion hybridizes to the monomorphic 3′ tail of the plurality of nucleic acids; b) a means for adding a monomorphic tail to the 3′ end of a first strand product; and, (c) a second oligo-dX-universal primer, wherein the second oligo-dX-universal primer comprises a 3′ oligo-dX portion and a second 5′ universal portion, wherein the second 5′ universal portion of the second oligo-dX-universal primer comprises a different sequence from the first 5′ universal portion of the first oligo-dX-universal primer, and wherein the oligo-dX portion of the second oligo-dX-universal primer comprises a nucleotide that is not complementary to the nucleotide of the oligo-dX portion of the first oligo-dX-universal primer. In some embodiments, the monomorphic tail of the 3′ ends of the first strand products comprise the same nucleotide as the monomorphic 3′ tail of the plurality of nucleic acids. In some embodiments, the monomorphic tail of the 3′ ends of the first strand products comprise adenine, and the monomorphic 3′ tail of the plurality of nucleic acids comprise adenine.

While the present teachings have been described in terms of these exemplary embodiments, the skilled artisan will readily understand that numerous variations and modifications of these exemplary embodiments are possible without undue experimentation. All such variations and modifications are within the scope of the current teachings.

Although the disclosed teachings have been described with reference to various applications, methods, kits, and compositions, it will be appreciated that various changes and modifications may be made without departing from the teachings herein and the claimed invention below. The foregoing examples are provided to better illustrate the disclosed teachings and are not intended to limit the scope of the teachings presented herein.

What is claimed is:

1. A method of amplifying a 3′ poly-A tail-containing nucleic acid in a sample comprising;
a) hybridizing a first oligo-dT-universal primer to the poly-A tail of the 3′ poly-A tail-containing nucleic acid, wherein the first oligo-dT-universal primer comprises a 3′ oligo-dT portion and a first 5′ universal portion, and wherein the 3′ oligo-dT portion hybridizes to the 3′ poly-A tail of the 3′ poly-A tail-containing nucleic acid;
b) extending the oligo-dT-universal primer in an extension reaction to form a first strand product comprising a 3′ end;
c) adding a poly-A tail to the 3′ end of the first strand product to form a two-tailed reaction product; and,
d) amplifying the two-tailed reaction product in a PCR to form an amplified 3′ poly-A tail-containing nucleic acid, wherein the PCR comprises the first oligo-dT-universal primer, and a second oligo-dT-universal primer, wherein the second oligo-dT-universal primer comprises a 3′ oligo-dT portion and a second 5′ universal portion, wherein the second 5′ universal portion of the second oligo-dT-universal primer comprises a different sequence than the first 5′ universal portion of the first oligo-dT-universal primer.

2. The method according to claim 1 comprising truncating the 3′ end of the first strand product with an exonuclease between b) and c).

3. The method according to claim 1 wherein the truncating comprises removing at least 100 nucleotides.

4. The method according to claim 1 wherein the 3′ oligo-dT portion of the first oligo-dT-universal primer comprises at least 20 dT's, and wherein the oligo-dT portion of the second oligo-dT-universal primer comprises at least 20 dT's.

5. The method according to claim 1 wherein the first 5′ universal portion of the first oligo-dT-universal primer comprises at least 10 nucleotides, and wherein the second 5′ universal portion of the second oligo-dT-universal primer comprises at least 10 nucleotides.

6. The method according to claim 1 wherein the sample comprises the entire transcriptome of 3′ poly-A tail-containing nucleic acids, the method further resulting in a plurality of amplified 3′ poly-A tail-containing nucleic acids.

7. The method according to claim 6 wherein the amplified 3′ poly-A tail-containing nucleic acids are further amplified in a primer-extension reaction comprising a promoter-linked primer to form a plurality of promoter-containing products, the method further comprising in vitro transcription of the promoter-containing products to form a plurality of in vitro transcription products, wherein the in vitro transcription comprises at least one labeled nucleotide.

8. The method according to claim 7 wherein the plurality of in vitro transcription products are analyzed on an array.

9. The method according to claim 6 wherein the first oligo-dT-universal primer or the second oligo-dT-universal primer further contains a promoter, the method further comprising in vitro transcription of the amplified 3′ poly-A tail-containing nucleic acid by a promoter-recognizing enzyme to form a plurality of in vitro transcription products, wherein the in vitro transcription comprises at least one labeled nucleotide.

10. The method according to claim 9 wherein the plurality of in vitro transcription products are analyzed on an array.

11. A method of amplifying a plurality of nucleic acids containing a monomorphic 3′ tail, comprising;
hybridizing a first oligo-dX-universal primer to the monomorphic 3′ tail of the plurality of nucleic acids, wherein the first oligo-dX-universal primer comprises a 3′ oligo-dX portion and a first 5′ universal portion, and wherein the 3′ oligo-dX portion hybridizes to the monomorphic 3′ tail of the plurality of nucleic acids;
extending the oligo-dX-universal primer in an extension reaction to form a plurality of first strand products comprising 3′ ends;
adding a monomorphic tail to the 3′ ends of the first strand products to form a plurality of two-tailed reaction products; and,
amplifying the plurality of two-tailed reaction products in a PCR, wherein the PCR comprises the first oligo-dX-universal primer, and a second oligo-dX-universal primer, wherein the second oligo-dX-universal primer comprises a 3′ oligo-dX portion and a second 5′ universal portion, wherein the second 5′ universal portion of the second oligo-dX-universal primer comprises a different sequence from the first 5′ universal portion of the first oligo-dx-universal primer, and wherein the oligo-dX portion of the second oligo-dX-universal primer comprises a nucleotide that is not complementary to the oligo-dX portion of the first oligo-dX-universal primer.

12. The method according to claim 11 wherein the monomorphic tail of the 3′ ends of the first strand products comprise the same nucleotide as the monomorphic 3′ tail of the plurality of nucleic acids.

13. The method according to claim 12 wherein the monomorphic tail of the 3′ ends of the first strand products comprise adenine, and the monomorphic 3′ tail of the plurality of nucleic acids comprise adenine.

14. A kit for amplifying a 3′ poly-A tail-containing nucleic acid in a sample comprising;
a) a first oligo-dT-universal primer, wherein the first oligo-dT-universal primer comprises a 3′ oligo-dT portion and a first 5′ universal portion, and wherein the 3′ oligo-dT portion hybridizes to the 3′ poly-A tail of a 3′ poly-A tail-containing nucleic acid;
b) a means for adding a poly-A tail to the 3′ end of a first strand product; and,
c) a second oligo-dT-universal primer, wherein the second oligo-dT-universal primer comprises a 3′ oligo-dT portion and a second 5′ universal portion, wherein the second 5′ universal portion of the second oligo-dT-universal primer comprises a different sequence than the first 5′ universal portion of the first oligo-dT-universal primer.

15. The kit according to claim 14 wherein the means for adding a poly-A tail to the 3′ end of the first strand product comprises a poly-A polymerase.

16. A kit for amplifying a plurality of nucleic acids containing a first monomorphic 3′ tail, comprising;
a) a first oligo-dX-universal primer complementary to the monomorphic 3′ tail of the plurality of nucleic acids, wherein the first oligo-dX-universal primer comprises a 3′ oligo-dX portion and a first 5′ universal portion, and wherein the 3′ oligo-dX portion hybridizes to the monomorphic 3′ tail of the plurality of nucleic acids;
b) a means for adding a monomorphic tail to the 3′ end of a first strand product; and,
c) a second oligo-dX-universal primer, wherein the second oligo-dX-universal primer comprises a 3′ oligo-dX portion and a second 5′ universal portion, wherein the second 5′ universal portion of the second oligo-dX-universal primer comprises a different sequence from the first 5′ universal portion of the first oligo-dX-universal primer, and wherein the oligo-dX portion of the second oligo-dX-universal primer comprises a nucleotide that is not complementary to the nucleotide of the oligo-dX portion of the first oligo-dX-universal primer.

17. The kit according to claim 16 wherein the monomorphic tail of the 3′ ends of the first strand products comprise the same nucleotide as the monomorphic 3′ tail of the plurality of nucleic acids.

18. The kit according to claim 16 wherein the monomorphic tail of the 3′ ends of the first strand products comprise adenine, and the monomorphic 3′ tail of the plurality of nucleic acids comprise adenine.
